(19) United States
(12) Patent Application Publication
(10) Pub. No.: US 2025/0253012 A1
(43) Pub. Date: Aug. 7, 2025

(54) Error Correction of Nucleic Acid Sequencing Reads

(71) Applicants: The Broad Institute, Inc., Cambridge, MA (US); Ultima Genomics, Inc., Fremont, CA (US); The General Hospital Corporation, Boston, MA (US)

(72) Inventors: Gad A. Getz, Belmont, MA (US); Julian Matsunaga Hess, Somerville, MA (US); Vasuki Narasimha Swamy, Cambridge, MA (US); Ilya Soifer, Tel Aviv (IL)

(73) Assignees: The Broad Institute, Inc., Cambridge, MA (US); Ultima Genomics, Inc., Fremont, CA (US); The General Hospital Corporation, Boston, MA (US)

(21) Appl. No.: 19/046,201

(22) Filed: Feb. 5, 2025

Related U.S. Application Data
(60) Provisional application No. 63/549,689, filed on Feb. 5, 2024.

(51) Int. Cl. G16B 40/20; G16B 30/10

(52) U.S. Cl. CPC G16B 40/20; G16B 30/10

(57) ABSTRACT

Error correction of nucleic acid sequencing reads is described. A machine learning model may be trained using aligned sequencing reads generated during a nucleic acid sequencing event, the aligned sequencing reads aligned to a reference sequence. The trained machine learning model may output predicted reference sequences for the aligned sequencing reads that have a position of mismatch with the reference sequence. The aligned sequencing reads may be selectively corrected according to whether the machine learning model predicts that the aligned sequencing reads are variants or sequencing errors based on the predicted reference sequences relative to the reference sequence.

### RELATED APPLICATION

This application claims priority to U.S. Provisional Patent Application Ser. No. 63/549,689, filed Feb. 5, 2024, entitled “Error Correction of Nucleic Acid Sequencing Reads,” the entire disclosure of which is hereby incorporated by reference herein in its entirety.

### BACKGROUND

Sequencing technologies have significantly advanced over the years, enabling researchers and clinicians to explore the genetic makeup, including genetic variations, of individuals with increased precision and/or at lower costs. Whole genome sequencing, for example, may be used to determine an entire sequence of a subject's genome, including non-coding regions, to provide a comprehensive view of the subject's genetic makeup. Sequencing technologies for whole genome sequencing and other types of sequencing enable study of the genetic basis of diseases, evolutionary relationships, and population-level variations. However, a cost of performing such sequencing using some techniques, such as sequencing by synthesis techniques, may be prohibitively high. While other sequencing techniques, such as sequencing by flow, may be less expensive, they may have higher sequencing error rates that can confound downstream analyses, such as variant calling.

### SUMMARY

Error correction of nucleic acid sequencing reads is described. A machine learning model may be trained using aligned sequencing reads generated during a nucleic acid sequencing event, the aligned sequencing reads aligned to a reference sequence. The trained machine learning model may output predicted reference sequences for the aligned sequencing reads that have a position of mismatch with the reference sequence. The aligned sequencing reads may be selectively corrected according to whether the machine learning model predicts that the aligned sequencing reads are variants or sequencing errors based on the predicted reference sequences relative to the reference sequence.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ error correction of nucleic acid sequencing reads.

FIG. 2 depicts an example implementation of the model training stage of the sequencing error correction module of FIG. 1 in greater detail.

FIG. 3 depicts an example implementation of the model use stage of the sequencing error correction module of FIG. 1 in greater detail.

FIG. 4 depicts an example architecture of the sequencing error learning model introduced in FIG. 1.

FIG. 5 depicts an example of sequencing reads having non-reference bases due to true variants.

FIG. 6 depicts an example of sequencing reads having non-reference bases due to true variants and random sequencing noise.

FIG. 7 depicts an example of sequencing reads having non-reference bases due to true variants and recurrent sequencing errors.

FIG. 8 depicts a workflow in an example implementation of variant calling by using the sequencing error correction module of FIG. 1 for learning and correcting sequencing artifacts.

FIG. 9 shows an example application of generating an error-corrected sequence by predicting the reference sequence of a read sequence.

FIG. 10 depicts an example procedure in which error correction of nucleic acid sequencing reads is performed for a sequencing event.

FIG. 11 depicts an example procedure in which error-corrected sequencing data are used for variant calling.

FIG. 12 illustrates an example system including various components of an example device that can be implemented as any type of computing device as described and/or utilized with reference to FIGS. 1-11 to implement embodiments of the techniques described herein.

### DETAILED DESCRIPTION

### Overview

Genome interpretation refers to a systematic analysis of the information encoded in an individual's genetic material, particularly the individual's deoxyribonucleic acid (DNA) sequence. This process involves identifying and annotating genetic variations, understanding their potential functional impacts, and correlating these findings with relevant biological and clinical knowledge. Genome interpretation aims to unveil the genetic basis of traits, diseases, and individual responses to therapeutic interventions, which contributes to the development of medicines (e.g., personalized medicines).

Genomic data are typically generated via DNA sequencing techniques. For instance, manual or automated DNA sequencing techniques may be used to determine a sequence of nucleotide bases in a DNA sample obtained from a subject. This process typically includes mapping sequencing reads, which are relatively short (e.g., hundreds or thousands of nucleotide bases) DNA fragments, to a reference genomic sequence (e.g., “reference genome”) in a process referred to as alignment. This results in a consensus sequence, e.g., the genomic sequence of the sample.

The genomic sequence of the sample may be compared to the reference genomic sequence to determine how the genomic sequence of the sample varies from that of the reference in a process referred to as variant calling. Variant calling includes identifying and characterizing genetic variations, or variants, in the genomic sequence of the sample in comparison to the reference genomic sequence. These variants can include single nucleotide polymorphisms (SNPs, where one nucleotide is changed to another at a specific position in the genome), insertions (e.g., the addition of one or more nucleotides at a specific position in the genome), and deletions (e.g., the removal of one or more nucleotides at a specific position in the genome).

However, imperfections in sequencing technologies can cause some bases to erroneously mismatch the reference in patterns that mimic variants such as point substitutions (e.g., SNPs) and insertions/deletions due to their occurrence at a same location across multiple sequencing reads. Current variant calling techniques may confound these recurrent mismatches to the reference as false positive genetic variants. This hampers the ability to accurately identify true genetic variants, which affects downstream analyses of disease etiology, population genetics, and so forth.

To overcome these problems, error correction of nucleic acid sequencing reads is disclosed herein. In accordance with the techniques described herein, sequencing data undergo error correction via a machine learning-based sequencing error suppression algorithm prior to variant calling, which increases an accuracy of the variant call by decreasing a detection of false positive genetic variants. The machine learning-based sequencing error suppression algorithm learns to identify recurrent patterns associated with sequencing errors and to distinguish these recurrent patterns from true genetic variants, thereby avoiding erroneously suppressing a signal from nonreference bases (e.g., bases in a sequencing read that do not match the reference genomic sequence at a given location) corresponding to true genetic variants. This error correction enables, for example, lower cost sequencing techniques that are more prone to sequencing errors to be used for lower cost, highly accurate and/or sensitive whole genome sequencing and variant calling.

In one or more implementations, the machine learning-based sequencing error suppression algorithm is trained on sequencing data generated during a sequencing run (e.g., sequencing event), making the machine learning-based sequencing error suppression algorithm able to identify and correct sequencing errors that are specific to that sequencing run. By way of example, the machine learning-based sequencing error suppression algorithm may be trained using a portion (e.g., less than a fraction of a percentage) of the sequencing data, with the reference genomic sequence providing ground truth outputs for the training. Via the training, the machine learning-based sequencing error suppression algorithm “learns” to predict the reference genomic sequence for nonreference bases occurring due to recurring sequencing artifacts. For instance, the sequences of nucleotides preceding and following the nonreference base provide context for patterns that are repeated when the nonreference base arises due to sequencing artifacts. In contrast, the machine learning-based sequencing error suppression algorithm is unable to predict the reference genomic sequence for true genetic variants with a high level of confidence because true genetic variants i) cause nonreference event bases at a much lower rate than artifacts and ii) do not occur in such patterns that are predictable given the amount of training data used. Accordingly, the machine learning-based sequencing error suppression algorithm is able to distinguish sequencing errors from true genetic variation based on whether the predicted reference genomic sequence matches the actual reference genomic sequence with high confidence. Such identified sequencing errors are corrected to the reference genomic sequence so that an impact of the sequencing errors on downstream analyses, such as variant calling, is reduced.

In some aspects, the techniques described herein relate to a system including: a sequencing error correction module configured to: train a machine learning model using a subset of aligned sequencing reads generated during a nucleic acid sequencing event, the aligned sequencing reads aligned to a reference sequence; output, by the trained machine learning model, a predicted reference sequence for a sequencing read of the aligned sequencing reads that has a position of mismatch with the reference sequence; and selectively correct the sequencing read based on the predicted reference sequence relative to the reference sequence.

In some aspects, the techniques described herein relate to a system including: a sequencing error correction module implemented in a non-transitory computer-readable storage medium and configured to: train a machine learning model using aligned sequencing reads generated during a nucleic acid sequencing event, the aligned sequencing reads aligned to a reference sequence; output, by the trained machine learning model, predicted reference sequences for the aligned sequencing reads that have a position of mismatch with the reference sequence; and selectively correct the aligned sequencing reads according to whether the machine learning model predicts that the aligned sequencing reads are variants or sequencing errors based on the predicted reference sequences relative to the reference sequence.

In some aspects, the techniques described herein relate to a system, wherein, to selectively correct the aligned sequencing reads based on the predicted reference sequence relative to the reference sequence, the sequencing error correction module is further configured to: update the position of mismatch in a first subset of the aligned sequencing reads in response to the predicted reference sequences matching the reference sequence at the position of mismatch with at least a threshold confidence; and maintain the position of mismatch in a second subset of the aligned sequencing reads in response to the predicted reference sequences not matching the reference sequence at the position of mismatch with at least the threshold confidence.

In some aspects, the techniques described herein relate to a system, wherein, to train the machine learning model using the aligned sequencing reads generated during the nucleic acid sequencing event, the sequencing error correction module is configured to: generate training data from the aligned sequencing reads, the training data including sequencing reads as model inputs and corresponding reference sequence segments as ground truth outputs; generate reference sequence predictions from the model inputs using the machine learning model; and adjust model parameters of the machine learning model based on a loss between the reference sequence predictions and the ground truth outputs.

In some aspects, the techniques described herein relate to a system, wherein the aligned sequencing reads used to train the machine learning model include a subset of less than 0.1% of the aligned sequencing reads generated during the nucleic acid sequencing event.

In some aspects, the techniques described herein relate to a system, wherein the machine learning model includes a neural network.

In some aspects, the techniques described herein relate to a system, wherein the neural network is a recurrent neural network.

In some aspects, the techniques described herein relate to a system, wherein the position of mismatch includes a substitution, an insertion, or a deletion.

In some aspects, the techniques described herein relate to a system, wherein the aligned sequencing reads include long reads, and wherein the sequencing error correction module is further configured to: subdivide the long reads into smaller read windows of 3000 bases or fewer.

In some aspects, the techniques described herein relate to a system, further including: a variant calling module configured to: output a variant call based on one or more differences between the selectively corrected aligned sequencing reads and the reference sequence.

In some aspects, the techniques described herein relate to a method including: receiving a sequencing alignment for a nucleic acid sequencing event, the sequencing alignment including a plurality of sequencing reads aligned to a reference sequence; training a machine learning model to predict a reference sequence segment for respective sequencing reads using, as training data, a subset of the plurality of sequencing reads and the reference sequence; and generating an error-corrected sequencing alignment based on the reference sequence and outputs of the trained machine learning model.

In some aspects, the techniques described herein relate to a method, wherein training the machine learning model to predict the reference sequence segment for the respective sequencing reads using, as the training data, the subset of the plurality of sequencing reads and the reference sequence includes: inputting the subset of the plurality of sequencing reads into the machine learning model; receiving, as an output of the machine learning model, predicted reference sequence segments for the subset; comparing the predicted reference sequence segments to corresponding segments of the reference sequence using a loss function; and adjusting model parameters of the machine learning model in a direction that reduces a loss calculated by the loss function.

In some aspects, the techniques described herein relate to a method, wherein generating the error-corrected sequencing alignment based on the reference sequence and the outputs of the trained machine learning model includes: identifying sequencing reads of the plurality of sequencing reads having a mismatched base with the reference sequence; inputting the identified sequencing reads into the machine learning model; receiving, as an output of the machine learning model, the predicted reference sequence segment for respective input sequencing reads; and selectively correcting the mismatched base with the reference sequence based on the predicted reference sequence segment relative to the reference sequence.

In some aspects, the techniques described herein relate to a method, wherein selectively correcting the mismatched base with the reference sequence based on the predicted reference sequence segment relative to the reference sequence includes: updating the mismatched base to be the reference sequence in response to the predicted reference sequence segment matching the reference sequence at a position of the mismatched base with at least a threshold probability; or maintaining the mismatched base in response to the predicted reference sequence segment not matching the reference sequence at the position of the mismatched base with at least the threshold probability.

In some aspects, the techniques described herein relate to a method, wherein the training data exclude sequencing reads aligned to genomic locations having single nucleotide polymorphism sites with a relative population-level frequency above a threshold percentage.

In some aspects, the techniques described herein relate to a method, wherein the machine learning model is trained to predict reference sequence segments that match a corresponding sequence segment of the reference sequence for sequencing reads that have a recurrent sequencing error, the recurrent sequencing error causing a sequence of a respective sequencing read to mismatch with the corresponding sequence segment of the reference sequence.

In some aspects, the techniques described herein relate to a method, wherein the recurrent sequencing error occurs at a same sequence position across multiple sequencing reads of the plurality of sequencing reads.

In some aspects, the techniques described herein relate to a method including: receiving nucleic acid sequencing data generated for a biological sample, the nucleic acid sequencing data including a plurality of sequencing reads; aligning the plurality of sequencing reads with a reference sequence; and generating an error-corrected sequence alignment based on nucleotide predictions output by a machine learning model, the machine learning model trained using a subset of the plurality of sequencing reads as model inputs and corresponding reference sequence segments of the reference sequence as ground truth outputs.

In some aspects, the techniques described herein relate to a method, wherein generating the error-corrected sequence alignment based on the nucleotide predictions output by the machine learning model includes: identifying a sequencing read of the plurality of sequencing reads having a position of mismatch with the reference sequence; predicting, via the machine learning model, a reference sequence segment for the sequencing read, the predicted reference sequence segment including a sequence of nucleotide predictions; and comparing a nucleotide prediction of the predicted reference sequence segment to an actual nucleotide of the reference sequence at the position of mismatch.

In some aspects, the techniques described herein relate to a method, wherein generating the error-corrected sequence alignment based on the nucleotide predictions output by the machine learning model further includes: correcting the sequencing read to include the actual nucleotide of the reference sequence at the position of mismatch in response to the nucleotide prediction matching the actual nucleotide; or maintaining a sequenced nucleotide of the sequencing read at the position of mismatch in response to the nucleotide prediction not matching the actual nucleotide.

In some aspects, the techniques described herein relate to a method, wherein generating the error-corrected sequence alignment is further based on probability scores output by the machine learning model, and wherein the machine learning model is further trained using covariates corresponding to the subset of the plurality of sequencing reads as additional model inputs.

In the following discussion, an example environment is first described that may employ the techniques described herein. Example implementation details and procedures are then described which may be performed in the example environment as well as other environments. Consequently, performance of the example procedures is not limited to the example environment and the example environment is not limited to performance of the example procedures.

### Example Environment

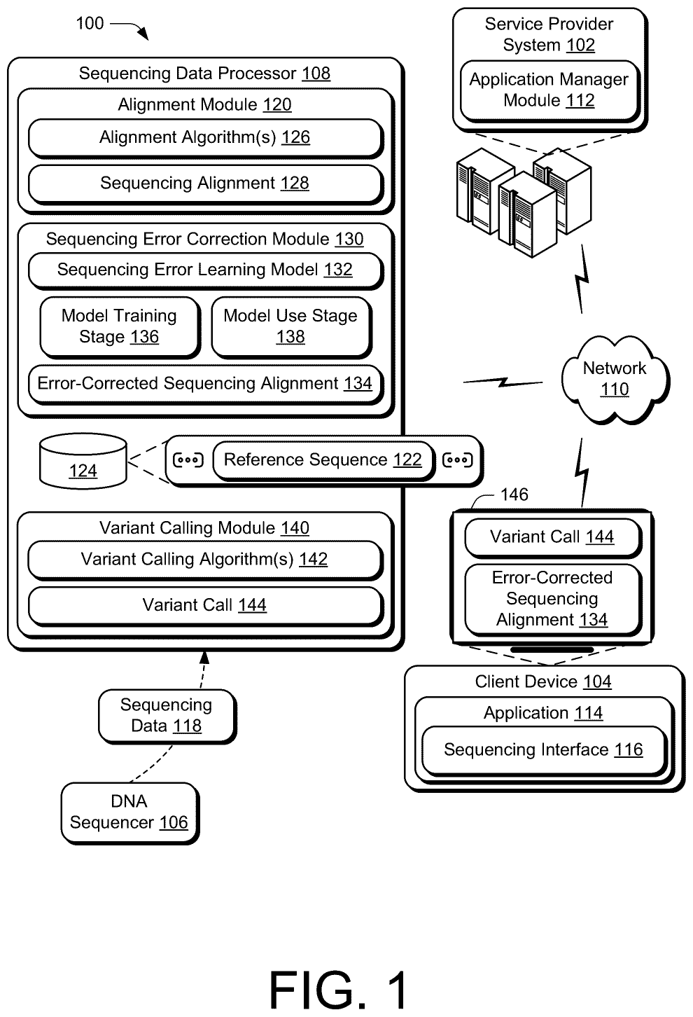

FIG. 1 is an illustration of an environment 100 in an example implementation that is operable to employ error correction of nucleic acid sequencing reads as described herein. The illustrated environment 100 includes a service provider system 102, a client device 104, a DNA sequencer 106, and a sequencing data processor 108 that are communicatively coupled, one to another, via a network 110. Although functionality of the sequencing data processor 108 is illustrated as separate from the service provider system 102, the client device 104, and the DNA sequencer 106, this functionality may be incorporated as part of the service provider system 102, the client device 104, and/or the DNA sequencer 106, further divided among other entities, and so forth. By way of example, an entirety of or portions of the functionality of the sequencing data processor 108 may be incorporated as part of the DNA sequencer 106. Additionally or alternatively, an entirety of or portions of the client device 104 may be incorporated as part of the DNA sequencer 106.

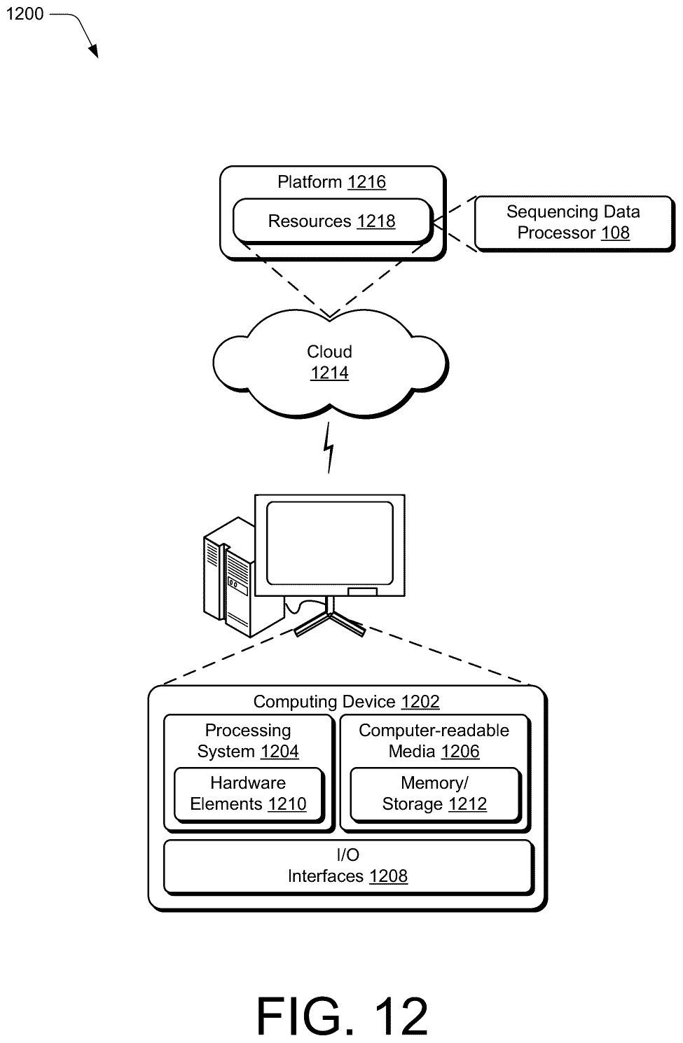

Computing devices that are usable to implement the service provider system 102, the client device 104, and the sequencing data processor 108 may be configured in a variety of ways. A computing device, for instance, may be configured as a desktop computer, a laptop computer, a mobile device (e.g., assuming a handheld configuration such as a tablet or mobile phone), and so forth. Thus, the computing device may range from full resource devices with substantial memory and processor resources (e.g., personal computers, game consoles) to a low-resource device with limited memory and/or processing resources (e.g., mobile devices). Additionally, a computing device may be representative of a plurality of different devices, such as multiple servers utilized to perform operations “over the cloud,” as further described in relation to FIG. 12.

The service provider system 102 is illustrated as including an application manager module 112 that is representative of functionality to provide access to the sequencing data processor 108 to a user of the client device 104 via the network 110. The application manager module 112, for instance, may expose content or functionality of the sequencing data processor 108 that is accessible via the network 110 by an application 114 of the client device 104. The application 114 may be configured as a network-enabled application, a browser, a native application, and so on, that exchanges data with the service provider system 102 via the network 110. The data can be employed by the application 114 to enable the user of the client device 104 to communicate with the service provider system 102, such as to receive application updates and features when the service provider system 102 provides functionality to manage the application 114.

In the context of the described techniques, the application 114 includes functionality to input parameters for a sequencing event as well as to analyze data generated by the sequencing event. In the illustrated example, the application 114 includes a sequencing interface 116 that is implemented at least partially in hardware of the client device 104 for facilitating communication between the client device 104 and the sequencing data processor 108. By way of example, the sequencing interface 116 includes functionality to receive inputs to the sequencing data processor 108 from the client device 104 (e.g., from a user of the client device 104) and output information, data, and so forth from the sequencing data processor 108 to the client device 104, as will be further elaborated herein.

The sequencing event includes determining an order of nucleotides (e.g., adenine, thymine or uracil, cytosine, and guanine) in a sample of nucleic acids, such as derived from a biological sample. The order of nucleotides is referred to herein as a “sequence.” The nucleotides are also referred to as “bases.” Although the sequencing event will be described herein with respect to deoxyribonucleic acid (DNA) sequencing, and more particularly genomic sequencing, it is to be appreciated that the techniques described herein may be adapted for sequencing other types of nucleic acids, such as ribonucleic acid (RNA). By way of example, sequencing can be performed using genomic DNA, complementary DNA (cDNA) derived from RNA transcripts, or RNA as a template.

The DNA sequencer 106 is configured to produce sequencing data 118 that is analyzed by the sequencing data processor 108 to determine the order of nucleotides in a sample. In at least one implementation, the sequencing data 118 comprise a text-based file format, such as FASTQ files that store both nucleotide sequence information and quality scores for the bases in a sequencing read. In variations, the sequencing data 118 comprise another type of file format. The DNA sequencer 106 may use one of a plurality of sequencing techniques to produce the sequencing data 118. By way of example, the DNA sequencer 106 may use a short read sequencing technique that produces sequence fragments typically ranging from approximately 10 bases to approximately 1000 bases and more typically from approximately 50 bases to approximately 800 bases. Sequence fragments produced via short read sequencing techniques are also referred to herein as “short reads.” Long read sequencing techniques produce sequence fragments that typically range from 2000 bases to 1,000,000 bases and more typically from 5000 bases to 800,000 bases in length. Sequence fragments produced via long read sequencing techniques are also referred to herein as “long reads.”

In general, short read sequencing techniques are higher throughput, which enables sequencing a plurality of nucleic acid fragments (e.g., DNA fragments) in parallel. One example of a short read sequencing technique is sequencing by synthesis, where complementary strands of nucleic acid fragments are synthesized during the sequencing process. Fluorescence detection is used to identify a newly incorporated nucleotide base-by-base. Another example of a short read sequencing technique is sequencing by flow. In sequencing by flow, rather than detecting each newly incorporated nucleotide base-by-base, homopolymers of nucleotides (e.g., stretches of sequences where consecutive nucleotides are the same, such as “AAA” or “GGGG”) are detected, with an intensity of a fluorescent signal indicating how many bases are in the homopolymer. Sequencing by flow is higher throughput and thus has a lower cost than sequencing by synthesis. However, sequencing by flow may be more prone to base call errors than sequencing by synthesis. Examples of long read sequencing techniques include single molecule real-time sequencing and nanopore sequencing.

Regardless of the sequencing technique used, the sequencing data processor 108 receives the sequencing data 118 and determines a sequence (e.g., a consensus sequence) of the nucleotides in the sample therefrom based at least in part on an output of an alignment module 120. The alignment module 120 is representative of functionality for performing read alignment of the sequencing data 118. Read alignment, also referred to simply as “alignment,” involves mapping the sequence fragments (e.g., reads) to locations in the genome using a reference sequence 122. The reference sequence 122 may be selected from a variety of nucleic acid sequences against which a sequence of a sample can be compared for determining an order of the nucleotides in the sample as well as variants therein, as will be elaborated below. The reference sequence 122 is a reference genome or a portion thereof. In one or more implementations, the sequencing data processor 108 includes or otherwise accesses a storage device 124 storing the reference sequence 122. The storage device 124 may store one or more other reference sequences in addition to the reference sequence 122, as indicated by ellipses in FIG. 1. By way of example, different reference sequences may correspond to different sample types or may come from a pangenome, which includes several high-quality, curated assemblies of individual genomes that may be represented jointly as a graph. As such, the reference sequence 122 is selected based on its similarity to the sample evaluated via the sequencing event, at least in some implementations. Moreover, the reference sequence 122 may include a combination of more than one individual reference sequence. By way of example, the reference sequence 122 may be a curated representation of an average population-level genome of an organism (e.g., the average human genome) or a portion of the average population-level genome.

In at least one implementation, the alignment module 120 is configured to map the sequencing data 118 to the reference sequence 122 via one or more alignment algorithms 126 to generate a sequencing alignment 128, also referred to herein as aligned reads. The one or more alignment algorithms 126 include functionality for finding an alignment that increases (e.g., maximizes) a similarity between a read and the reference sequence 122 using a scoring system that considers possible mismatches between the reference sequence 122 and the sequencing data 118, e.g., insertions, deletions, and point substitutions. The sequencing alignment 128 comprises sequence fragments (e.g., reads) that have been successfully mapped to the reference sequence 122.

Typically, the sequencing alignment 128 is used to determine the consensus sequence of the nucleotides in the sample. By way of example, at each position in the sequencing alignment 128, the nucleotide present in the majority of read sequences is chosen for the consensus sequence at that position. This process may involve counting the occurrences of each base at a specific position. However, the sequencing data 118, and thus the sequencing alignment 128, may have base call errors. A base call error occurs when a detected base at a given position in a read sequence does not reflect an actual base at that position. In at least one implementation, base call errors include insertion errors, deletion errors, and substitution errors, which result in mismatches between the reference sequence 122 and the sequencing read. The base call errors may be collectively referred to as “sequencing errors” or “sequencing artifacts” herein. An insertion error occurs when an extra nucleotide is incorrectly added to the sequence. As an illustrative example, if the true DNA sequence is “ATCG,” a base call of “ATGCG” includes an insertion error of a G between the T and the C. A deletion error occurs when a nucleotide is omitted or skipped during sequencing. Continuing with the above example where the true DNA sequence is “ATCG,” a base call of “ACG” includes a deletion error of the T between the A and the C. A substitution error occurs when the wrong nucleotide is identified in place of the correct one. Again using the example where the true DNA sequence is “ATCG,” a base call of “AGCG” includes a substitution error where the T has been changed to a G. It is to be appreciated that more than one consecutive nucleotide may be mismatched between the read sequence and the reference sequence 122, such as the insertion of two or more consecutive nucleotides, the deletion of two or more consecutive nucleotides, and/or point substitutions at two or more consecutive nucleotides. Moreover, a given read sequence may have more than one position of mismatch with the reference sequence 122. In various scenarios, sequencing errors result in a determined base at a particular sequence location not matching the reference sequence 122, which has implications not only for an accuracy of the resulting consensus sequence, but also for downstream analyses such as variant calling.

“Variant calling” refers to the identification and/or characterization of genetic variations, or “variants,” in a sequence determined for a sample (e.g., an individual's genome) when compared to a reference sequence (e.g., a reference genome). These variants may include short variants, such as single nucleotide polymorphisms (SNPs, where one nucleotide is changed to another at a specific position in the genome), insertions (e.g., the addition of one or more nucleotides at a specific position in the genome), and deletions (e.g., the removal of one or more nucleotides at a specific position in the genome). Insertions and deletions may be collectively referred to as “INDELs.” Additionally or alternatively, the variants may include larger, structural variations, such as copy number variants (CNVs, where a segment of DNA ranging from kilobases to megabases in size is duplicated or deleted), inversions (e.g., where a segment of DNA is reversed in orientation), INDELs involving larger segments (e.g., more than 50 nucleotides), translocations (e.g., where a segment of DNA is moved from one location to another, often involving the exchange of genetic material between non-homologous chromosomes), and replacements (e.g., where a segment of DNA is replaced or substituted by another, which may include additional changes such as insertions, duplications, or other rearrangements). As may be appreciated from the above, sequencing errors mimic short variants, such as SNPs and smaller INDELs. As such, sequencing errors and true variants both lead to mismatches between the sequencing alignment 128 and the reference sequence 122.

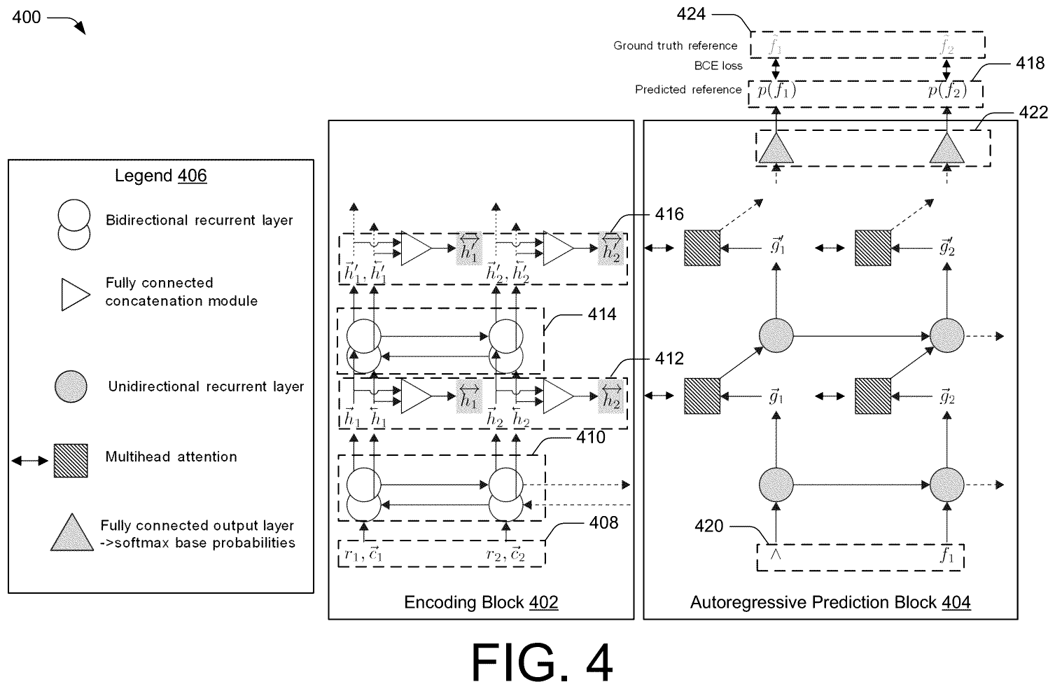

Thus, in accordance with the techniques described herein, the sequencing data processor 108 includes a sequencing error correction module 130. The sequencing error correction module 130 is representative of the functionality to distinguish between erroneously mismatched bases and true variants. The sequencing error correction module 130 uses a sequencing error learning model 132 to generate an error-corrected sequencing alignment 134 from the sequencing alignment 128, such as will be elaborated herein. The sequencing error learning model 132 is a machine learning model that is trained based on a portion (e.g., a small subset) of the aligned reads in the sequencing alignment 128 during a model training stage 136 and performs error correction on the sequencing alignment 128 during a model use stage 138. In at least one implementation, the sequencing error learning model 132 is configured to perform sequence-to-sequence translation, such as by outputting a predicted reference sequence corresponding to an input read. By way of example, the sequencing error learning model 132 is a recurrent neural network (RNN) with an attention mechanism. However, in variations, the sequencing error learning model 132 additionally or alternatively includes another type of model, such as another type of neural network, a long short-term memory (LSTM) model, a gated recurrent unit (GRU) model, an encoder-decoder model, a RNN without an attention mechanism, a hidden Markov model (HMM), and/or a transformer model, just to name a few. An example architecture will be described herein with respect to FIG. 4.

As mentioned above, the sequencing error learning model 132 is trained on the portion of the aligned reads in the sequencing alignment 128. As such, training data are generated by the sequencing error correction module 130 in the model training stage 136 from the sequencing alignment 128, and the sequencing error learning model 132 is specifically trained for each separate sequencing event. By way of example, the sequencing error correction module 130 selects a fixed number or percentage of reads to use as the training data. The training data, for instance, may comprise less than 0.05% of the sequencing alignment 128, such as a value in a range from 0.01% to 0.03%. In the context of short reads, the training data may comprise between 50,000 and 800,000 reads, or between 75,000 and 400,000 reads, or between 100,000 and 200,000 reads. When the sequencing data 118 comprise long reads, the long reads may be subdivided into smaller read windows of several kilobases (e.g., 1000-3000 bases) or fewer (e.g., 800-1000 bases, or 150-300 bases) to generate the training data. It is also contemplated that an entirety of the sequencing alignment 128 may be used as the training data. However, using the entirely of the sequencing alignment 128 may train the sequencing error learning model 132 to erroneously suppress mismatches due to true genetic variants, which is undesirable.

In at least one implementation, the model training stage 136 uses self-supervised learning, where the sequencing error correction module 130 relies on the alignment to identify which sites are reference (e.g., sites that match the reference sequence 122 at a given sequence location) or nonreference (e.g., sites that do not match the reference sequence 122 at the given sequence location) to train the sequencing error learning model 132. In at least one variation, the model training stage 136 uses semi-supervised learning, where sites of common polymorphisms are excluded from the training data, in addition to or as an alternative to the self-supervised learning. In other variations, the model training stage 136 additionally or alternatively uses supervised learning (where nonreference events in each sequencing read are explicitly labeled as true variant or artifact). However, the supervised learning technique relies on manual labeling (e.g., as performed by humans), which is time-consuming and may not be practical. By way of example, a human user may not know which nonreference events are true variants and which are artifacts.

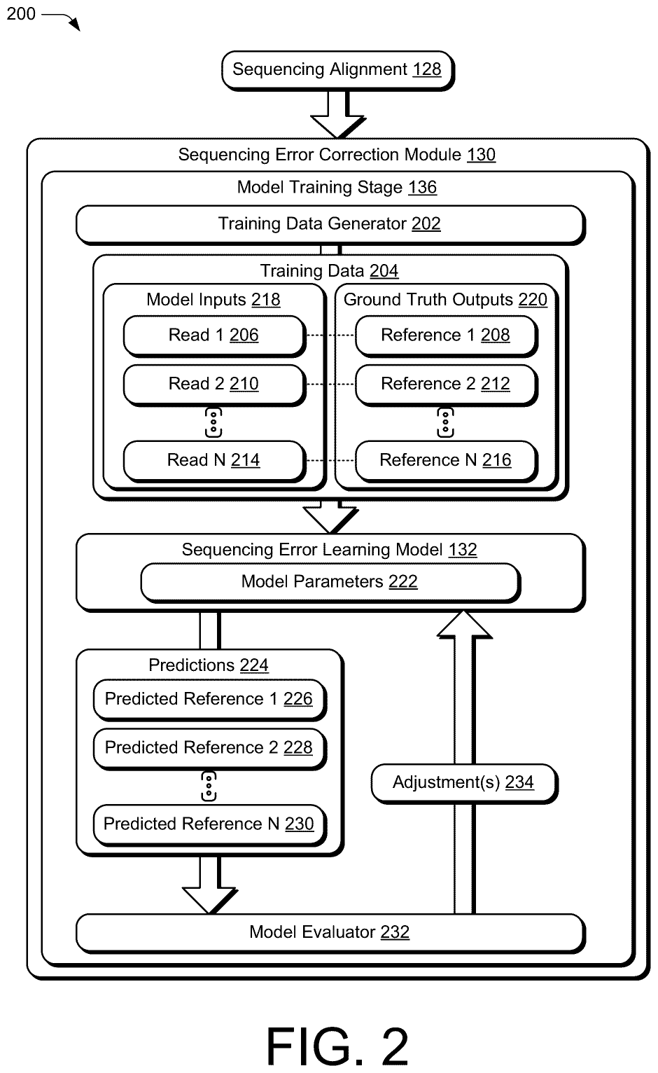

In at least one implementation, the sequencing error learning model 132 is randomly initialized and trained during the model training stage 136 using the training data generated from the sequencing alignment 128. During the training, the sequencing error learning model 132 “learns” to predict the actual reference sequence for a given input read sequence. Because true variants are random, whereas sequencing errors that would confound variant calling have a higher probability at specific positions and/or within recurrent patterns, the sequencing error learning model 132 is unable to accurately predict with a high level of confidence the actual reference sequence for true variants given the amount of training data used in the training process. Thus, the sequencing error learning model 132 “learns” to predict the actual reference sequence for reads having recurrent sequencing errors and not for true variants. Additional details regarding the model training stage 136 will be described herein with respect to FIG. 2.

Notably, the specific recurrent patterns and artifact rate may vary from sequencing event to sequencing event. As such, training the sequencing error learning model 132 on the sequencing alignment 128 enables the sequencing error learning model 132 to accurately distinguish between mismatches due to sequencing errors and those due to true variants in a manner that is specific to a given sequencing event. By way of example, a sequencing error learning model trained using reads from a first sequencing event may be less accurate at identifying sequencing errors in sequencing reads generated via a second sequencing event. However, it is contemplated that in certain circumstances, it may be desirable to use a sequencing error learning model trained with data from one sequencing event to identify and/or suppress errors for reads from another sequencing event. As such, the data used to train the model and the sequencing event for which the model is used should not be limited unless otherwise specifically stated.

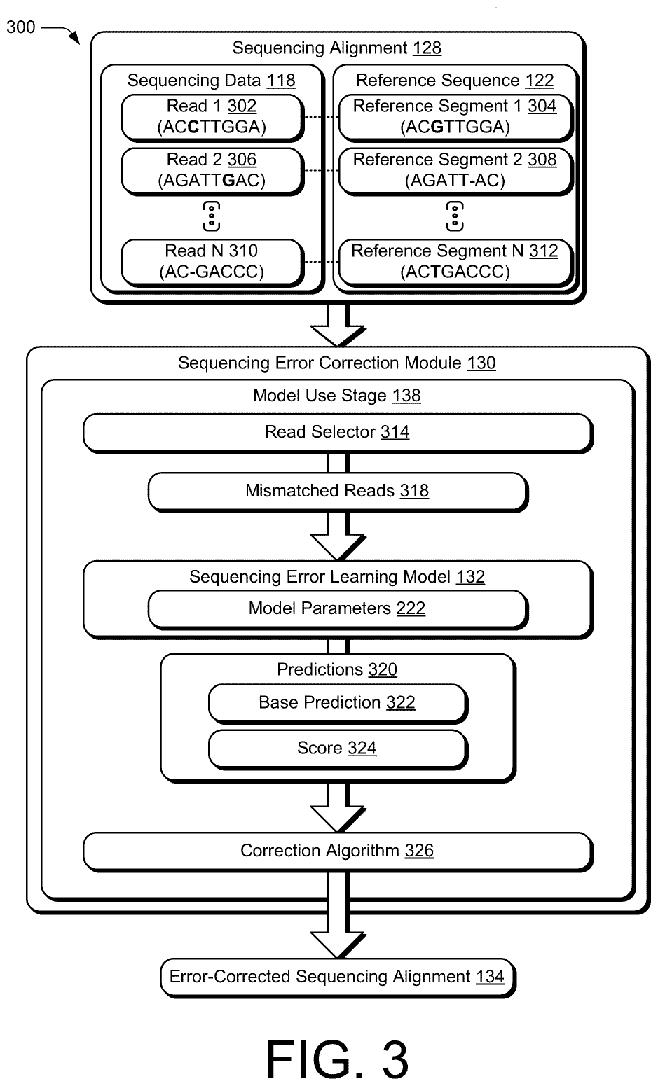

Once the sequencing error learning model 132 is trained, the aligned sequencing reads of the sequencing alignment 128 are evaluated during the model use stage 138 to identify mismatches due to sequencing errors. As will be elaborated herein with respect to FIG. 3, during the model use stage 138, the sequencing error learning model 132 predicts a reference sequence to which a read sequence is aligned, which is then compared to the actual reference sequence. Because the sequencing error learning model 132 learns sequence contexts that induce to recurrent errors, the sequencing error learning model 132 accurately predicts the reference sequence (e.g., the predicted reference sequence matches the actual reference sequence) when mismatches between the read and the reference sequence are due to sequencing errors. In contrast, the sequencing error learning model 132 is unable to accurately predict the reference sequence for true variants (e.g., the predicted reference sequence does not match the actual reference sequence). The sequencing error learning model 132 thus identifies locations of sequencing errors, which may be suppressed by correcting the mismatch to the reference sequence to generate the error-corrected sequencing alignment 134.

The sequencing data processor 108 further includes a variant calling module 140 representative of the functionality for determining genomic differences between the error-corrected sequencing alignment 134 and the reference sequence 122. By way of example, after the sequencing error correction module 130 uses the sequencing error learning model 132 to identify and correct mismatches due to sequencing errors, variants are detected by identifying positions where the error-corrected sequencing alignment 134 differs from the reference sequence 122. In at least one implementation, the variant calling module 140 includes one or more variant calling algorithms 142 to generate a variant call 144 using statistical and/or computational methods. The variant call 144 includes an indication of one or more variants, including small variant(s) and/or structural variant(s), that are determined to be present in the error-corrected sequencing alignment 134 compared to the reference sequence 122.

In at least one implementation, the one or more variant calling algorithms 142 do not perform error correction to distinguish between true genetic variants and sequencing errors/artifacts. In variations, the one or more variant calling algorithms 142 include machine learning-based methods to classify whole sets of reads as supporting a true variant or a sequencing error/artifact. However, in contrast to the techniques described herein, these machine learning-based variant callers rely on hand-curated ground truth labels to distinguish variants from artifacts. This supervised learning approach also makes these techniques specific to the sequencing platform, variant type (e.g., germline versus somatic), and sample type (e.g., DNA versus RNA) for which they were trained. Unlike these error-correcting variant callers, the sequencing error correction module 130 described herein does not rely on manually curated ground truth labeling and is broadly applicable to different sequencing technologies and sample types.

The client device 104 is shown displaying, via a display device 146, the error-corrected sequencing alignment 134, or a portion thereof, as well as the variant call 144. By way of example, the display device 146 may display a portion of the error-corrected sequencing alignment 134 as a string of characters representing the sequence of nucleotides in the portion. Additionally, or alternatively, the display device 146 may display the error-corrected sequencing alignment 134 as a visual representation of the reads aligned with the reference sequence 122 along with an indication of a nucleotide identified at a specific location. The variant call 144 may be displayed by the display device 146 as a visual representation of genomic location(s) where variant(s) are present and/or as a list of detected variant(s) and their genomic location(s). It is to be appreciated that the variant call 144 and the error-corrected sequencing alignment 134 are also stored in memory, in a single data file or multiple data files, for subsequent access.

In this way, the sequencing data processor 108 enables true genetic variants to be accurately identified, for example, even using lower-cost, higher throughput sequencing techniques that are more prone to sequencing errors and artifacts. As a result, sequencing and monetary resources are more efficiently utilized without sacrificing an amount and quality of genomic information obtained. Alternatively, the techniques described herein can be employed to improve sequencing techniques that are more expensive and/or lower throughput.

### Sequencing Error Correction Using Machine Learning

FIG. 2 depicts an example implementation 200 of the model training stage 136 of the sequencing error correction module 130 of FIG. 1 in greater detail. The example implementation 200 includes, from FIG. 1, the sequencing alignment 128 and the sequencing error correction module 130, including the sequencing error learning model 132 and the model training stage 136.

In the example implementation 200, the sequencing error correction module 130 includes a training data generator 202. The sequencing error correction module 130, and thus the training data generator 202, receives the sequencing alignment 128 as input, and the training data generator 202 generates training data 204 based on the sequencing alignment 128. As discussed above with respect to FIG. 1, the training data 204 comprise a portion of the sequencing alignment 128, such as a pre-programmed number (e.g., 100,000-200,000) or percentage (e.g., 0.01-0.1%) of the aligned sequencing reads of the sequencing alignment 128. The training data 204 are represented in FIG. 2 as a first read 206 (e.g., “read 1”) that is aligned with a first reference sequence segment 208 (e.g., “reference 1”), a second read 210 (e.g., “read 2”) that is aligned with a second reference sequence segment 212 (e.g., reference 2″), and an n^{th} read 214 (e.g., “read N”) that is aligned with an n^{th} reference sequence segment 216 (e.g., “reference N”). Ellipses indicate that additional read and reference sequence segment pairs may be included in the training data 204. In this example, the first reference sequence segment 208 provides a ground truth reference for the first read 206, the second reference sequence segment 212 provides a ground truth reference for the second read 210, and the n^{th} reference sequence segment 216 provides a ground truth reference for the n^{th} read 214.

As such, the training data 204 is divided into model inputs 218, which include the sequencing reads, and ground truth outputs 220, which include the corresponding reference sequence segments to which the sequencing reads are aligned. For example, because the majority of read/reference mismatches are due to error and not true variants, the sequencing error correction module 130 assumes that the read is a corrupted version of the reference sequence segment, making the corresponding reference the ground truth for each read. Moreover, because the sequencing error learning model 132 is configured to predict a reference sequence for a given input sequencing read, the reference sequence segment to which the input sequencing read is aligned is the true sequence that the sequencing error learning model 132 attempts to predict (e.g., the ground truth).

It is to be appreciated that the sequencing error correction module 130 may receive other inputs in addition to the sequencing alignment 128, such as quality metrics (e.g., covariates), which provide additional context for the sequencing error learning model 132. For instance, a lower quality score indicates a higher likelihood of a base call being inaccurate.

In at least one implementation, areas of the genome that commonly include single nucleotide polymorphisms are not included in the training data 204. By way of example, the training data generator 202 may filter or otherwise exclude sequencing reads aligned to regions having common single nucleotide polymorphism sites, such as those having a relative population-level frequency above a threshold percentage.

The sequencing error learning model 132 includes model parameters 222. The model parameters 222 include, for example, weights and variables that are randomly initialized. In at least one variation, however, the model parameters 222 are pre-trained, and the model training stage 136 retrains and/or refines the model parameters 222 for the sequencing data 118 of the particular sequencing run. The training process performed during the model training stage 136 iteratively adjusts the model parameters 222 in a direction that reduces (e.g., minimizes) a loss function, as elaborated below.

In the present example, the sequencing error learning model 132 receives the model inputs 218 and outputs predictions 224. The predictions 224 include a first predicted reference sequence 226 (“predicted reference 1”) for the first read 206, a second predicted reference sequence 228 (“predicted reference 2”) for the second read 210, and an n^{th} predicted reference sequence 230 (“predicted reference N”) for the n^{th} read 214. A model evaluator 232 compares the predictions 224 to the ground truth outputs 220 to generate one or more adjustments 234 to the model parameters 222. The model evaluator 232, for instance, is representative of the functionality of the sequencing error correction module 130 for calculating a loss, performing backpropagation (e.g., backpropagation through time), and adjusting the model parameters 222. By way of example, the model evaluator 232 compares the first predicted reference sequence 226 to the first reference sequence segment 208, the second predicted reference sequence 228 to the second reference sequence segment 212, and the n^{th} predicted reference sequence 230 to the n^{th} reference sequence segment 216 using a loss function that calculates a discrepancy between the predicted reference sequence and the corresponding ground truth reference sequence segment, e.g., nucleotide by nucleotide. As a non-limiting example, the loss function is a binary cross-entropy loss function. Performing backpropagation may include computing gradients with respect to the model parameters 222 that indicate how much each parameter contributed to the discrepancy. The model evaluator 232 may use an optimization algorithm, such as stochastic gradient descent or a derivative thereof, to update the model parameters 222 in a direction that reduces (e.g., minimizes) the loss. The process is repeated iteratively, with the sequencing error learning model 132 outputting updated predictions 224 using the updated model parameters 222, until convergence is reached, for example, and the model training stage 136 ends.

FIG. 3 depicts an example implementation 300 of the model use stage 138 of the sequencing error correction module 130 of FIG. 1 in greater detail. The example implementation 300 includes, from FIG. 1, the sequencing alignment 128 and the sequencing error correction module 130, including the sequencing error learning model 132 and the model use stage 138. The implementation 300 further includes the model parameters 222 introduced with respect to FIG. 2.

As described with respect to FIG. 1, the sequencing alignment 128 includes the sequencing data 118 aligned to the reference sequence 122. By way of example, a first read 302 (e.g., “read 1”) is aligned with a first reference sequence segment 304 (e.g., “reference segment 1”), a second read 306 (e.g., “read 2”) is aligned with a second reference sequence segment 308 (e.g., reference segment 2″), and an n^{th} read 310 (e.g., “read N”) is aligned with an n^{th} reference sequence segment 312 (e.g., “reference segment N”). Ellipses indicate that additional reads and reference sequence segments are included. It is to be appreciated that a plurality of reads may be aligned with a same segment of the reference sequence 122. As a non-limiting example illustrated in FIG. 3, the first read 302 has a sequence ACCTTGGA while the first reference sequence segment 304 has a sequence ACGTTGGA, indicating a substitution mismatch at the third position; the second read 306 has a sequence AGATTGAC while the second reference sequence segment 308 has a sequence AGATT-AC, indicating an insertion mismatch at the sixth position; and the n^{th} read 310 has a sequence AC-GACCC while the n^{th} reference sequence segment 312 has a sequence ACTGACCC, indicating a deletion mismatch at the third position.

During the model use stage 138, the sequencing error correction module 130 receives the sequencing alignment 128. It is to be appreciated that the sequencing error correction module 130 may also receive additional inputs, such as covariates. A read selector 314 identifies sequencing reads that have mismatches with the reference sequence 122. For example, a mismatch is present when a read has a nucleotide at a particular position that differs from what is present in the corresponding position of the reference sequence 122 or fails to have the nucleotide present in the corresponding reference sequence 122, such as due to an insertion, deletion, or substitution. The read selector 314 outputs the identified reads, represented in FIG. 3 as mismatched reads 318, for analysis by the sequencing error learning model 132, which has been trained to identify recurrent mismatches that are due to sequencing artifacts (e.g., errors). By way of example, analyzing reads having mismatches with the reference segment rather than an entirety of the reads in the sequencing alignment 128 reduces computing resource consumption, as nucleotides that match the reference at a given position will not be identified for error correction. It is to be appreciated that when the mismatched reads 318 include long reads, the long reads may be subdivided into smaller read windows of several kilobases (e.g., 1000-3000 bases) or fewer (e.g., 800-1000 bases, or 150-300 bases) around the position of mismatch.

The sequencing error learning model 132 outputs predictions 320 that include a base prediction 322 and a score 324. By way of example, based on the training of the model parameters 222 using the training data 204, the sequencing error learning model 132 predicts a most likely reference sequence for a given input read of the mismatched reads 318. The base prediction 322 corresponds to a predicted nucleotide at the mismatched position (e.g., the position where there is a mismatch between the sequencing data 118 and the reference sequence 122), and the score 324 corresponds to a confidence or probability value regarding the base prediction 322 being the reference base. If the input read has a learned sequencing error (e.g., as learned via the model training stage 136), the sequencing error learning model 132 outputs the reference nucleotide as the base prediction 322, and the score 324 has a high confidence or probability. In contrast, if the input read does not have a learned sequencing error, such as when the mismatched position reflects a true variant or random noise, the sequencing error learning model 132 does not output the reference nucleotide as the base prediction 322 and/or the score 324 has a low confidence or probability.

A correction algorithm 326 receives the predictions 320 and outputs the error-corrected sequencing alignment 134. By way of example, the correction algorithm 326 may use a score threshold to determine whether to use the read base or the reference base in the error-corrected sequencing alignment 134 at a given mismatched position. When the score 324 has high confidence (e.g., the score 324 is greater than or equal to the score threshold), the reference base is selected. That is, the mismatch is determined, by the sequencing error learning model 132, to be a sequencing error in response to the score 324 being greater than or equal to the score threshold, and so the error-corrected sequencing alignment 134 includes the nucleotide of the reference sequence 122 at that position. In contrast, when the score 324 has low confidence (e.g., the score 324 is less than the score threshold), the read base is selected for the error-corrected sequencing alignment 134. That is, in response to the score 324 being less than the score threshold, the mismatch is not recognized by the sequencing error learning model 132 as a recurrent sequencing error, and so the sequencing data 118 is not corrected. In this way, true variants are not suppressed because the sequencing error learning model 132 is unable to “learn” the randomness of true genetic variants using the model training stage 136.

Continuing with the illustrative example sequences of FIG. 3, a reference sequence prediction of ACGTTGGA for the first read 302 matches the first reference sequence segment 304, and so the first read 302 is corrected to have the sequence ACGTTGGA in the error-corrected sequencing alignment 134. A reference sequence prediction of AGATTAAC for the second read 306 does not match the second reference sequence segment 308, and so the first reference sequence segment 304 is maintained as the sequence AGATTGAC in the error-corrected sequencing alignment 134. A reference sequence prediction of ACTGACCC for the n^{th} read 310 matches the n^{th} reference sequence segment 312, and so the n^{th} read 310 is corrected to have the sequence ACTGACCC.

FIG. 4 depicts an example architecture 400 of the sequencing error learning model 132 introduced in FIG. 1. The architecture 400 includes an encoding block 402 and an autoregressive prediction block 404. A legend 406 is also provided that defines symbols depicted in the architecture 400. It is to be appreciated that the architecture 400 is one example implementation of an architecture that may be used for the sequencing error learning model 132, and variations are possible without departing from the scope of the described techniques.

The encoding block 402 receives an input sequence 408 at a first bidirectional recurrent layer 410. In the depicted example, the input sequence 408 includes read sequence bases (r) and covariates (c). By way of example, r₁ corresponds to a nucleotide at a first position of the read sequence, and c₁ corresponds to a covariate at the first position. In at least one implementation, the covariate is given as vector. Similarly, r₂ corresponds to a nucleotide at a second position of the read sequence, and c₂ corresponds to a covariate at the second position. Dashed arrows indicate that additional bases and corresponding covariates may be included in the input sequence 408. In at least one implementation, the covariates include quality scores of the base calls that influence model output probabilities.

Via the bidirectional recurrent layers, the encoding block 402 processes the input sequence 408 in both forward and backward directions in order to identify patterns in the input sequence 408. The forward direction includes processing the input sequence 408 from left to right (e.g., from r₁ to r₂), and the backward direction includes processing the input sequence 408 from right to left (e.g., from r₂ to r₁).

The first bidirectional recurrent layer 410 computes first hidden states (h) for respective positions in the input sequence 408. A hidden state is an internal vector that captures and summarizes information about the sequence observed up to that position, thus providing context for the sequence up to that position. The first bidirectional recurrent layer 410 computes first forward hidden states and first backward hidden states based on the processing direction of the input sequence 408. By way of example, a first forward hidden state and a first backward hidden state h₁ are computed based on the inputs r₁ and c₁, and a first forward hidden state and a first backward hidden state h₂ are computed based on the inputs r₂ and c₂. A first concatenation 412 of the forward hidden state and the backward hidden state is performed to determine a final hidden state for a respective position using a fully connected concatenation module. The final hidden state summarizes a preceding sequencing of a given position in the input sequence 408 and a following sequence at the given position in the input sequence 408.

A second bidirectional recurrent layer 414 computes a second forward hidden state and a second backward hidden state (h′) for each position in the input sequence 408 based on the input sequence 408 and the first hidden state, which undergo a second concatenation 416 similar to the process described above for the first concatenation 412. This process is repeated for additional layers of the encoding block 402, as indicated by dashed vertical arrows.

The autoregressive prediction block 404 predicts a nucleotide of a reference sequence to which the input sequence 408 is aligned based on the hidden states calculated by the encoding block 402 using unidirectional recurrent layers and multihead attention. The predicted nucleotides are output as predictions 418. This process uses predictions from previous positions, as indicated by an offset predicted sequence 420. For example, in the offset predicted sequence 420, a predicted reference nucleotide at the first position, f₁, is input into unidirectional recurrent layers predicting a reference nucleotide at the second position, f₂. The incorporation of the multihead attention enables the sequencing error learning model 132 to consider different parts of the input sequence 408, even from both past and future contexts, when making the predictions 418.

In the autoregressive prediction block 404, attentional vectors (g) are determined based on the hidden states calculated in the encoding block 402 in combination with the context from the offset predicted sequence 420. By way of example, gi is computed for the first position, and gi is computed for the second position for each unidirectional recurrent layer. The attentional vectors are fed into a softmax layer 422 to produce the predictions 418. The predictions 418 further include a probability associated with the predicted reference nucleotide. During the model training stage 136, the predictions 418 are compared to ground truth references 424 for a given nucleotide position, and a loss (e.g., a binary cross-entropy loss) is calculated.

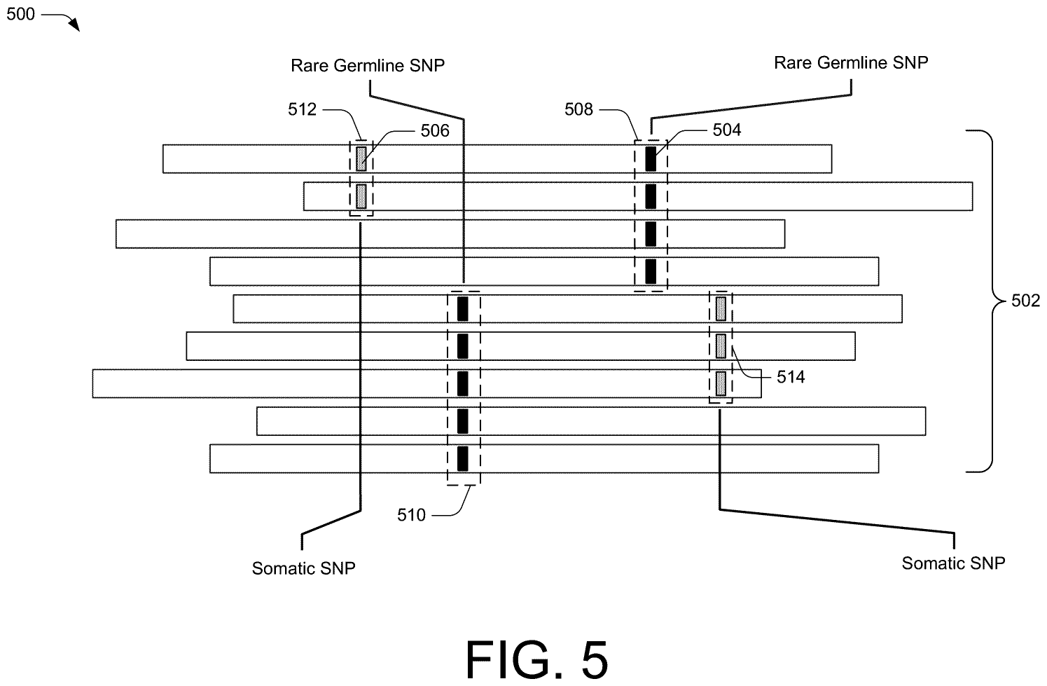

Examples of the ability of the sequencing error learning model 132 to identify and correct recurrent mismatches due to sequencing artifacts will now be described by comparing FIGS. 5-7. Referring first to FIG. 5, an example 500 of sequencing reads having non-reference bases due to true variants is shown. The example 500 includes aligned reads 502. It is to be appreciated that the example 500 is simplified, and a size and quantity of the aligned reads 502 is not to scale. The example 500 includes germline single nucleotide polymorphisms (SNPs) 504, indicated by solid filled boxes, and somatic SNPs 506, indicated by shaded boxes. Only one germline SNP 504 and only one somatic SNP 506 are labeled in FIG. 5 for illustrative clarity. The germline SNPs 504 are variations in a single nucleotide that occur at a specific position in the genome that are inherited, whereas the somatic SNPs 506 arise during the lifetime of an organism. For instance, the somatic SNPs 506 contribute to approximately 1 out of 1,000,000 non-reference bases (e.g., nucleotides that do not match the reference sequence 122, not shown in FIG. 5, at a given position), making them rarer than the germline SNPs 504. In general, the germline SNPs 504 are found on one or both alleles in every cell, whereas the somatic SNPs 506 are cell specific. The example 500 includes a first germline SNP location 508, a second germline SNP location 510, a first somatic SNP location 512, and a second somatic SNP location 514.

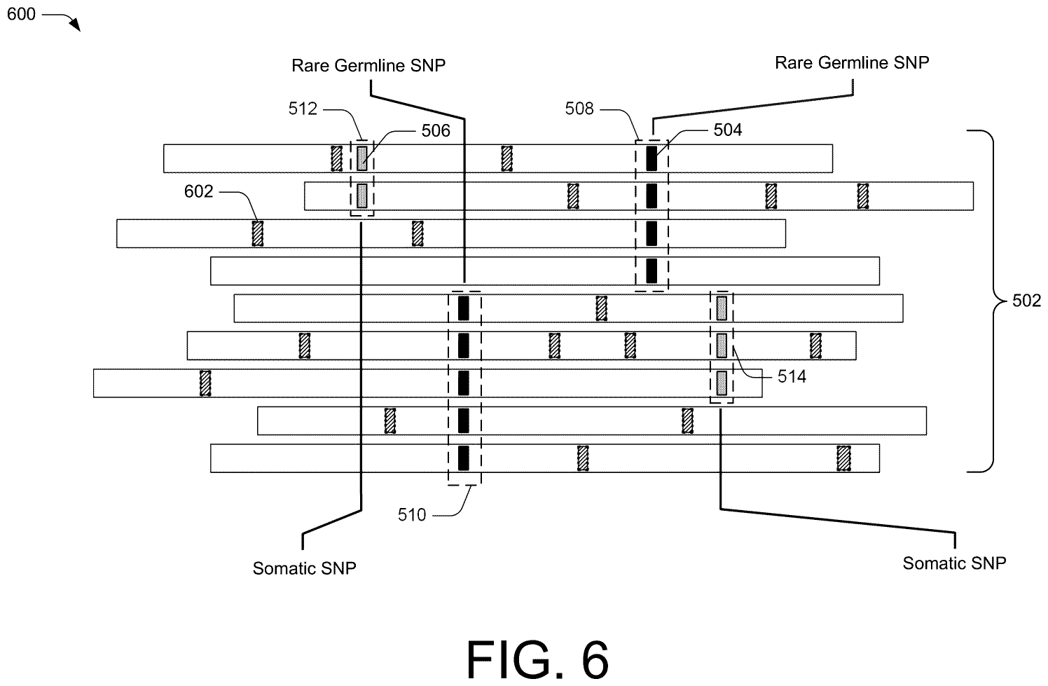

FIG. 6 depicts an example 600 of sequencing reads having non-reference bases due to true variants and random sequencing noise. The example 600 shows sequencing artifacts 602 on the aligned reads 502. The sequencing artifacts 602 are indicated by diagonally shaded boxes, only one of which is labeled for illustrative clarity. In the example 600, the sequencing artifacts 602 are randomly distributed throughout the aligned reads 502 such that the sequencing artifacts 602 are not aligned with respect to each other. Randomly distributed sequencing artifacts 602 may have little impact on variant calling because the non-reference nucleotide does not repeat throughout the sequencing data at a given position. For example, a non-reference base that is due to a randomly distributed sequencing artifact may not be selected for a consensus sequence used for the variant call.

It is to be appreciated that in the example 600, the sequencing artifacts 602 comprise a majority of the non-reference bases. In general, sequencing artifacts may occur at higher frequency than true variants, with the frequency depending on the type of sequencing technology used. By way of example, errors may occur during sequencing by synthesis in approximately 1 out of 1000 sequenced bases, while sequencing by flow error rates may be approximately 1 out of 150 sequenced bases.

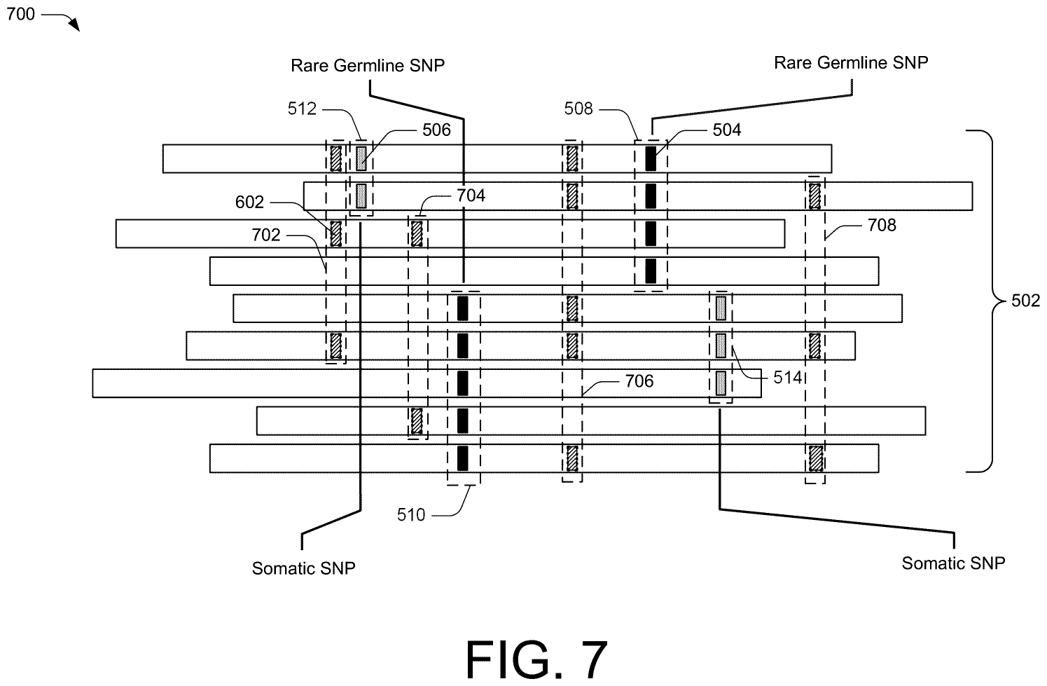

FIG. 7 depicts an example 700 of sequencing reads having non-reference bases due to true variants and recurrent sequencing errors. Similar to the example 600 of FIG. 6, the example 700 shows sequencing artifacts 602 on the aligned reads 502. However, unlike in the example 600, the sequencing artifacts 602 independently occur at the same genomic position across multiple reads, which mimics the distribution of the germline SNPs 504 and the somatic SNPs 506. For instance, the example 700 includes a first position 702, a second position 704, a third position 706, and a fourth position 708 with sequencing artifacts 602 that occur on multiple individual reads of the aligned reads 502.

Because the sequencing artifacts 602 arise independently (e.g., one read does not influence another read) at the same genomic position, the sequencing artifacts 602 are a function of the sequence itself, which enables the error to be predicted according to the techniques described herein. In this way, the sequencing error learning model 132 is able to accurately predict recurrent sequencing artifacts that may affect variant calling while ignoring random sequencing artifacts that do not impact variant calling. Because the germline SNPs 504 and the somatic SNPs 506 are rare relative to the sequencing artifacts 602, an amount of training data used during the model training stage 136 is insufficient for the sequencing error learning model 132 to “learn” to accurately predict true variants. Moreover, the sequence context of recurrent sequencing artifacts is more structured (and thus predictable) than that of true variants, which further enhances an ability of the sequencing error learning model 132 to distinguish between recurrent sequencing errors and true variants.

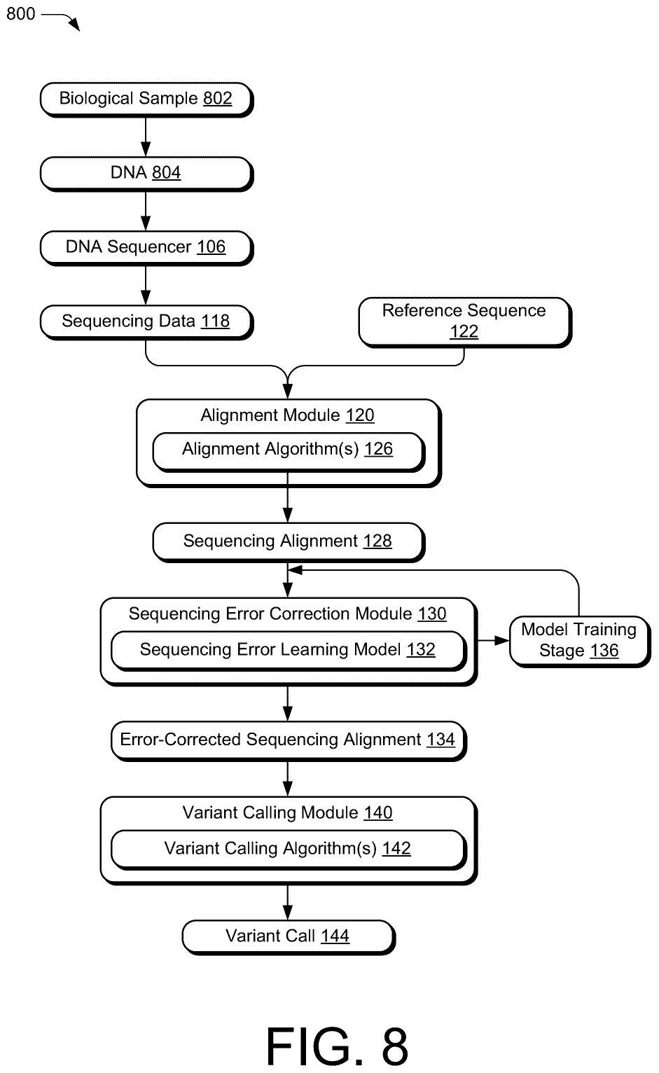

FIG. 8 depicts a workflow 800 in an example implementation of variant calling by the sequencing data processor 108 of FIG. 1 using the sequencing error correction module 130 for learning and correcting sequencing artifacts. For instance, the workflow 800 outlines a sequencing pipeline for a sequencing event with reference to components previously introduced with respect to FIG. 1.

A biological sample 802 is processed to prepare a nucleic acid sample, shown in FIG. 8 as DNA 804. By way of example, the DNA 804 (or another nucleic acid) is isolated from the biological sample 802 using a DNA extraction technique. The biological sample 802 comprises, for example, blood, tissue, saliva, or another source of cells from an organism (e.g., individual) of interest. Moreover, the DNA 804 may be prepared for sequencing according to a protocol specified by a type of sequencing technique being used. This includes, for example, breaking the nucleic acid into fragments.

Sequencing is performed by the DNA sequencer 106 to produce the sequencing data 118 from the DNA 804. In at least one implementation, the DNA sequencer 106 employs fluorescence-based detection to determine an order of nucleotides in fragments of the DNA 804. The sequencing data 118 comprises reads, which are ordered combinations of nucleotides, for the nucleic acid fragments.

As discussed above with respect to FIG. 1, the alignment module 120 receives the sequencing data 118 and maps the reads to the reference sequence 122 using the one or more alignment algorithms 126, resulting in the sequencing alignment 128. It is to be appreciated that as used herein, the term “align” and its conjugates is not limited to an exact 1:1 alignment between sequences. Rather, alignment is accomplished with a degree of accuracy that is adequate or desired based on its intended purpose (e.g., to determine a consensus sequence with a sufficient confidence and/or accuracy).

A portion of the aligned reads in the sequencing alignment 128 are used by the sequencing error correction module 130 to train the sequencing error learning model 132 during the model training stage 136. As described with respect to FIG. 2, the model training stage 136 uses a small subset of the sequencing alignment 128 as training data so that the sequencing error learning model 132 “learns” to distinguish recurrent sequencing errors and not true variants or random errors. Once the sequencing error learning model 132 is trained, the sequencing error correction module 130 evaluates the sequencing alignment 128 to identify positions where a read does not match the reference sequence 122. These reads having mismatches are evaluated by the sequencing error learning model 132, which identifies recurrent sequencing errors by being able to accurately predict the reference sequence 122 at the position of the mismatch. The recurrent sequencing errors are corrected by changing the mismatched nucleotide(s) to those of the reference sequence 122 at the corresponding position, resulting in the error-corrected sequencing alignment 134. In at least one variation, the error-corrected sequencing alignment 134 includes an error-corrected consensus sequence in addition to or as an alternative to the corrected aligned reads. For instance, the error-corrected consensus sequence is reconstructed by assembling the aligned error-corrected reads into a longer, contiguous sequence based on overlapping portions of the aligned error-corrected reads.

The error-corrected sequencing alignment 134 (or the error-corrected consensus sequence) is compared to the reference sequence 122 by the variant calling module 140 to output the variant call 144 via the one or more variant calling algorithms 142. The variant call 144 may be used for downstream applications, such as population studies of germline or somatic variants, hereditary studies, tracking of de novo mutations in disease states (e.g., cancer), identifying new cancer drivers, etc.

Having discussed example details of the techniques for error correction of nucleic acid sequencing reads, consider now an example to illustrate usage of the techniques.

### Example Application

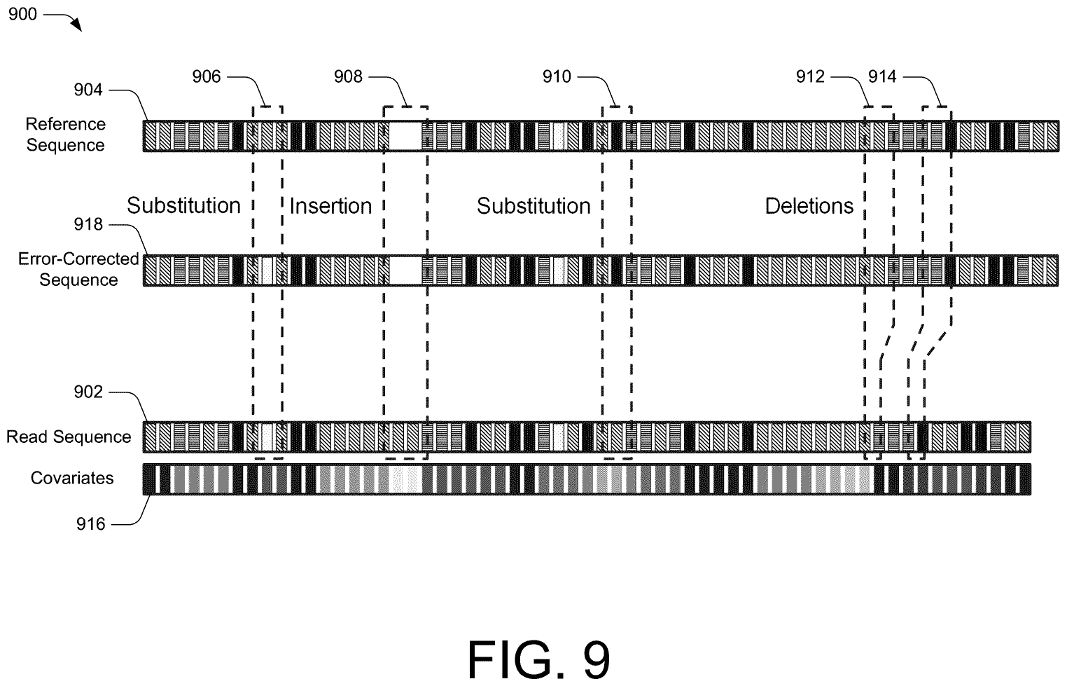

FIG. 9 shows an example application 900 of generating an error-corrected sequence by predicting the reference sequence of a read sequence.

The depicted example shows a read sequence 902 and a reference sequence 904, with different box shading representing different nucleotides. For example, diagonal shading represents adenine (A), horizontal shading represents thymine (T), dark shading represents cytosine (C), and light shading represents guanine (G). In the depicted example, the read sequence 902 varies from the reference sequence 904 at five genomic positions. In other words, the read sequence 902 has five mismatches with the reference sequence 904. A substitution is present a first position 906, where the read sequence 902 includes a G (light shaded box) instead of the A (diagonally shaded box) of the reference sequence 904. An insertion is present at a second position 908, where the read sequence 902 includes two A nucleotides (diagonally shaded boxes) that are not present in the reference sequence 904. Another substitution is present at a third location 910, where the read sequence 902 includes an A (diagonally shaded box) instead of the C (dark shaded box) of the reference sequence 904. A deletion is present at a fourth location 912, where the read sequence 902 is missing an A (diagonally shaded box) that is present in the reference sequence 904. Another deletion is present at a fifth location 914, where the read sequence 902 is missing a T (horizontally shaded box) that is present in the reference sequence 904.

After the sequencing error learning model 132 is trained using the reference sequence 904 as a ground truth, the read sequence 902 and corresponding covariates 916 (e.g., quality scores) are input into the sequencing error learning model 132 (not shown in FIG. 9), which results in an error corrected sequence 918. By way of example, at the first position 906, although the reference sequence 904 includes an A, the sequencing error learning model 132 is unable to predict the A. As such, the G from the read sequence 902 is maintained in the error corrected sequence 918 and represents a true variant. At the second position 908, the sequencing error learning model 132 accurately predicts that the two A nucleotides are not present in the reference sequence 904. As such, the two A nucleotides present in the read sequence 902 at the second position 908 are not included in the error corrected sequence 918. At the third location 910, the sequencing error learning model 132 accurately predicts the C of the reference sequence 904 despite the A in the read sequence 902. As such, the C of the reference sequence 904 is used in the error corrected sequence 918. The sequencing error learning model 132 accurately predicts that an A is present in the reference sequence 904 at the fourth location 912 despite being deleted in the read sequence 902. As such, the A of the reference sequence 904 is included at the fourth location 912 of the error corrected sequence 918. Similarly, the sequencing error learning model 132 accurately predicts that a T is present in the reference sequence 904 at the fifth location 914 despite being deleted in the read sequence 902. Accordingly, the T of the reference sequence 904 is included in the error corrected sequence 918 at the fifth location 914.

It is to be appreciated that the application 900 is a simplified illustrative example and is not meant to denote sequence patterns that give rise to the various mismatches described above.

Having discussed example details of the techniques for error correction of nucleic acid sequencing reads, consider now example procedures to illustrate additional aspects of the techniques.

### Example Procedures

This section describes example procedures for error correction of nucleic acid sequencing reads in one or more implementations. Aspects of the procedures may be implemented in hardware, firmware, software, or a combination thereof. The procedures are shown as a set of blocks that specify operations performed by one or more devices and are not necessarily limited to the orders shown for performing the operations by the respective blocks. In at least some implementations, the procedures are performed by a suitably configured device, such as the sequencing data processor 108 of FIG. 1.

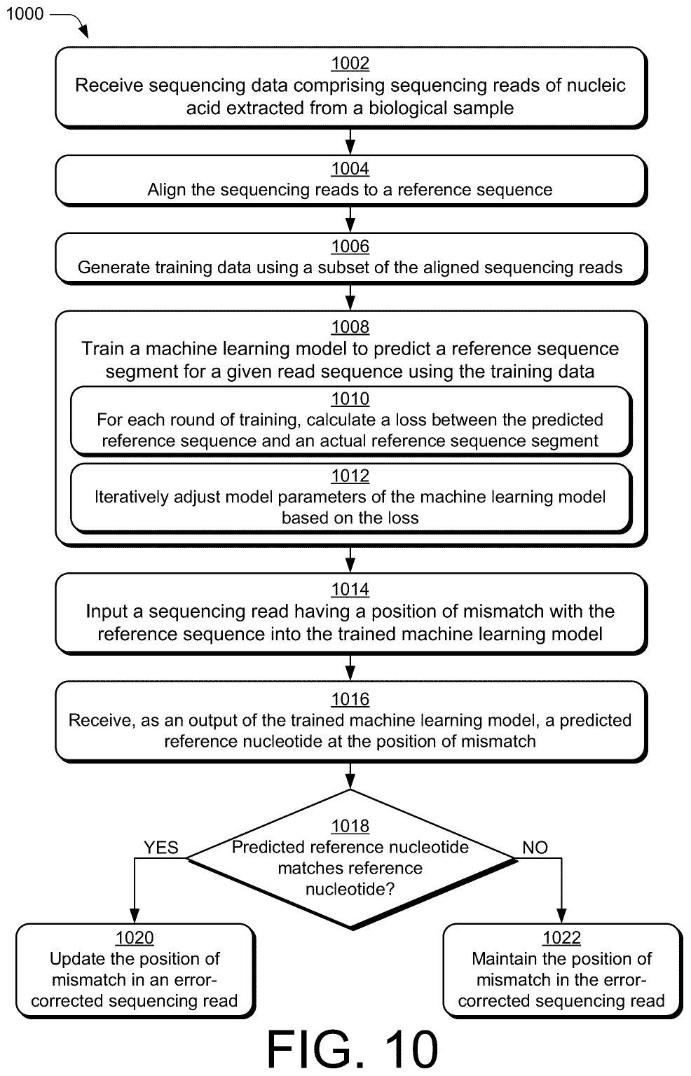

FIG. 10 depicts an example procedure 1000 in which error correction of nucleic acid sequencing reads is performed for a sequencing event.

Sequencing data comprising sequencing reads of nucleic acid extracted from a biological sample are received (block 1002). By way of example, the sequencing data (e.g., the sequencing data 118 of FIG. 1) are generated by a DNA sequencer (e.g., the DNA sequencer 106 of FIG. 1) and may comprise short read sequencing data or long read sequencing data depending on a specific DNA sequencing technique used. By way of example, the DNA sequencer may use a short read sequencing technique that produces sequence fragments typically ranging from approximately 10 bases to approximately 1000 bases and more typically from approximately 50 bases to approximately 800 bases. Alternatively the DNA sequencer may use a long read sequencing technique that produces sequence fragments that typically range from 2000 bases to 1,000,000 bases and more typically from 5000 bases to 800,000 bases in length. In at least one implementation, the sequencing includes whole genome sequencing. In one or more variations, the sequencing includes targeted sequencing, where a specific subset of genes or genomic regions are specifically targeted, or exome sequencing, where protein-coding regions of the genome are sequenced.

The sequencing reads are aligned to a reference sequence (block 1004). By way of example, an alignment module (e.g., the alignment module 120 of FIG. 1) uses one or more alignment algorithms to map the sequencing reads to locations in the genome using a reference sequence (e.g., a reference genome). The one or more alignment algorithms include functionality for finding an alignment that increases (e.g., maximizes) a similarity between a read and the reference sequence using a scoring system that considers possible insertions, deletions, and mismatches. Aligning the sequencing reads to the reference genome generates a sequencing alignment, which comprises sequence fragments (e.g., the sequencing reads) that have been successfully mapped to the reference sequence.

Training data are generated using a subset of the aligned sequencing reads (block 1006). By way of example, the training data may be selected by a sequencing error correction module (e.g., the sequencing error correction module 130 of FIG. 1) during a model training stage. By way of example, the sequencing error correction module selects a fixed number or percentage of sequencing reads to include in the training data. The training data, for instance, may comprise less than 0.1% of the sequencing reads or less than 0.05% of the sequencing reads, such as a value in a range from 0.01% to 0.03%. In the context of short read sequencing, the training data may comprise between 50,000 and 800,000 reads, or between 75,000 and 400,000 reads, or between 100,000 and 200,000 reads. When long read sequencing is used, the long reads may be subdivided into smaller read windows of several kilobases (e.g., 1000-3000 bases) or fewer (e.g., 800-1000 bases, or 150-300 bases) to generate the training data.

The selected sequencing reads provide model inputs of the training data. The training data further include ground truth outputs, which comprise corresponding reference sequence segments to which the sequencing reads are aligned. It is to be appreciated that the training data may include other inputs in addition to the selected sequencing reads, such as quality metrics (e.g., covariates), which provide additional context for the sequencing reads. For instance, a lower quality score indicates a higher likelihood of a base call being inaccurate within a sequencing read.

Moreover, in at least one implementation, areas of the genome that include common single nucleotide polymorphisms may not be selected for the training data. By way of example, sequencing reads aligned to regions having single nucleotide polymorphism sites with a relative population-level frequency above a threshold percentage may be excluded and/or filtered from the training data.

A machine learning model is trained to predict a reference sequence segment for a given read sequence using the training data (block 1008). By way of example, the machine learning model (e.g., the sequencing error learning model 132 of FIG. 1) is trained during the model training stage using self-supervised learning since the reference sequence provides the ground truth outputs. In at least one variation, the machine learning model is trained via another technique, such as unsupervised learning, supervised learning, and/or semi-supervised learning, in addition to or as an alternative to the self-supervised learning. However, self-supervised learning advantageously avoids tedious hand-labeling of data. Given that the machine learning model is trained specifically for the sequencing data of the biological sample, it may not be practical to provide manual ground truth labels for every sequenced biological sample.

In at least one implementation, the machine learning model begins with randomly initialized model parameters. Alternatively, the machine learning model is pre-trained, and the training includes retraining and/or refining the model parameters. During the training, the machine learning model “learns” to predict the reference sequence segment for a given input read sequence. In accordance with the techniques described herein, the training includes, for each round of training, calculating a loss between the predicted reference sequence and an actual reference sequence segment (block 1010) and iteratively adjusting model parameters of the machine learning model based on the loss (block 1012). By way of example, the machine learning model outputs base predictions for each nucleotide position of the reference sequence based on the input read sequence. The output predicted reference sequence is compared to a corresponding ground truth output, e.g., the actual reference sequence segment to which the input read sequence is aligned. The loss is calculated nucleotide by nucleotide to determine a discrepancy between the predicted reference sequence and the corresponding ground truth output. In at least one implementation, the loss is calculated via a binary cross-entropy loss function. Then, the model parameters are adjusted using an optimization algorithm, such as a stochastic gradient descent algorithm, to update the model parameters in a direction that reduces (e.g., minimizes) the loss. The process is repeated iteratively, with the machine learning model outputting updated predicted reference sequences using the updated model parameters, until convergence and/or a desired number of training rounds is reached.

Because true variants are random whereas sequencing errors that would confound variant calling have a higher probability at specific positions and/or within recurrent patterns, the machine learning model “learns” to accurately predict the actual reference sequence segment for recurrent sequencing errors, and not true variants, given the amount of training data used in the training process.

A sequencing read having position of mismatch with the reference sequence is input into the trained machine learning model (block 1014). For example, a mismatch is present when the sequencing read has a nucleotide at a particular position that differs from what is present in the corresponding position of the reference sequence, such as due to a substitution. As another example, a mismatch is present when the sequencing read is missing the nucleotide present at the corresponding position of the reference sequence (e.g., a deletion) or has an additional nucleotide that is not present in the reference sequence (e.g., an insertion).

A predicted reference nucleotide at the position of mismatch is received as an output of the trained machine learning model (block 1016). By way of example, the sequencing read having the position of mismatch is input into the trained machine learning model. The machine learning model predicts a most likely reference sequence for the input sequencing read. As a part of this, the predicted reference nucleotide at the position of mismatch is output. In at least one implementation, the machine learning model additionally outputs a score corresponding to a confidence or probability value regarding the predicted reference nucleotide at the position of mismatch.

It is determined if the predicted reference nucleotide matches a reference nucleotide of the reference sequence at the position of mismatch (block 1018). If the input sequencing read has a learned sequencing error (e.g., as learned during the model training) at the position of mismatch, the predicted reference nucleotide matches the reference nucleotide. If the input sequencing read does not have a learned sequencing error at the position of mismatch, the predicted reference nucleotide does not match the reference nucleotide.

In at least one implementation, the score is additionally used to determine if the predicted reference nucleotide matches the reference nucleotide at the position of mismatch. By way of example, the predicted reference nucleotide is considered to match the reference nucleotide in response to the predicted reference nucleotide having a same identity (e.g., adenine, thymine, guanine, or cytosine) and the score being at least a threshold confidence or probability. In contrast, the predicted reference nucleotide is not considered to match the reference nucleotide in response to the predicted reference nucleotide having a different identity and/or the score being less than the threshold confidence or probability.

If the predicted reference nucleotide matches the reference nucleotide, the position of mismatch is updated in an error-corrected sequencing read (block 1020). By way of example, the error-corrected sequencing read includes the reference nucleotide (or lack thereof), rather than the nucleotide of the sequencing read, at the position of mismatch. An error-corrected sequencing alignment may thus be generated that incorporates the error-corrected sequencing read for a plurality of sequencing reads having mismatches with the reference in order to suppress recurrent sequencing errors that mimic true variants.

If the predicted reference nucleotide does not match the reference nucleotide, the position of mismatch is maintained in the error-corrected sequencing read (block 1022). By way of example, the error-corrected sequencing read maintains the nucleotide of the sequencing read at the position of mismatch so that true variants are not suppressed. Although the position of mismatch remains unchanged, it is considered to be error-corrected because it has been evaluated by the machine learning model. In this way, the procedure 1000 selectively corrects the sequencing read based on the predicted reference nucleotide relative to the reference nucleotide at the position of mismatch.

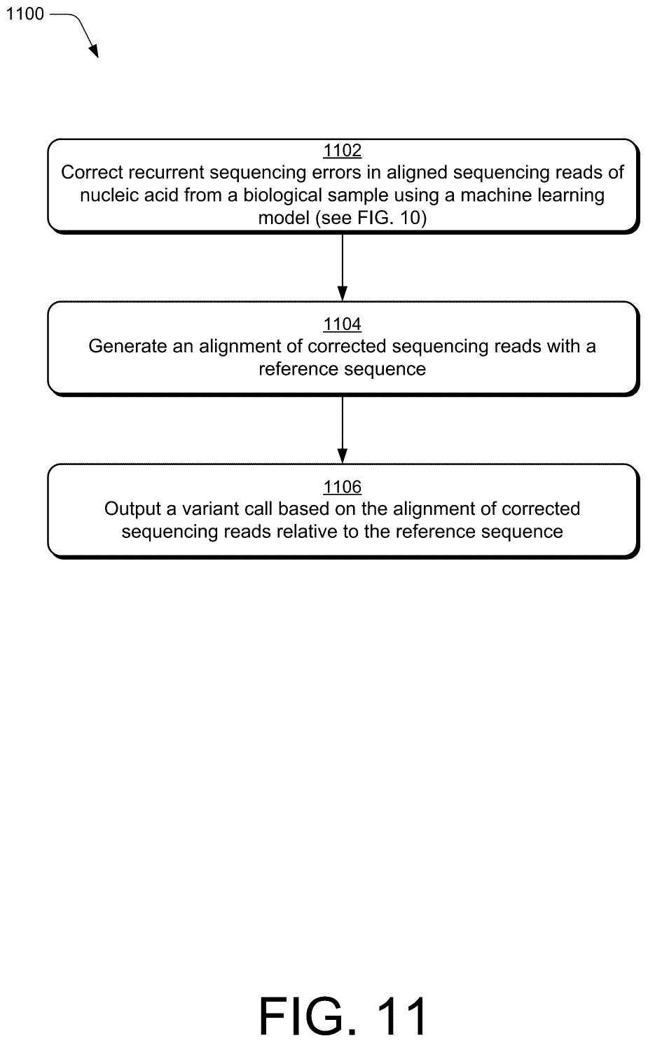

FIG. 11 depicts an example procedure 1100 in which error-corrected sequencing data is used for variant calling. In at least one implementation, the procedure 1100 is used in sequence with the procedure 1000 of FIG. 10.

Recurrent sequencing errors are corrected in aligned sequencing reads of nucleic acid from a biological sample using a machine learning model (block 1102). By way of example, the machine learning model described with respect to procedure 1000 of FIG. 10 is used to selectively correct positions of mismatch between the aligned sequencing reads and a reference sequence that correspond to the recurrent sequencing errors, which occur as a result of the sequencing process and the sequence context.

An alignment of corrected sequencing reads with the reference sequence is generated (block 1104). By way of example, the corrected sequencing reads are aligned with the reference sequence to generate a corrected sequencing alignment.

A variant call is output based on the alignment of the corrected sequencing reads relative to the reference sequence (block 1106). By way of example, a variant calling module (e.g., the variant calling module 140 of FIG. 1) compares the alignment of the corrected sequencing reads with the reference sequence and identifies positions where the alignment of the corrected sequencing reads differs from the reference sequence. In at least one implementation, the variant calling module uses one or more variant calling algorithms to identify short variants (e.g., SNPs or INDELs) and/or structural variants (e.g., CNVs, inversions, translocations, large deletions, and so forth) using statistical and/or computational methods. The variant call includes an indication of one or more variants, including small variant(s) and/or structural variant(s), that are determined to be present in the alignment of the corrected sequencing reads compared to the reference sequence. In at least one implementation, the one or more variant calling algorithms do not perform error correction to distinguish between true genetic variants and sequencing errors/artifacts. As such, by correcting the sequencing reads prior to the variant calling, an occurrence of identifying false positive genomic variants in the variant call is reduced. In this way, an accuracy of downstream analyses of disease etiology, population genetics, and so forth is increased.

Having described example procedures in accordance with one or more implementations, consider now an example system and device that can be utilized to implement the various techniques described herein.

### Example System and Device

FIG. 12 illustrates an example system generally at 1200 that includes an example computing device 1202 that is representative of one or more computing systems and/or devices that may implement the various techniques described herein. This is illustrated through inclusion of the sequencing data processor 108. The computing device 1202 may be, for example, a server of a service provider, a device associated with a client (e.g., a client device), an on-chip system, and/or any other suitable computing device or computing system.

The example computing device 1202 as illustrated includes a processing system 1204, one or more computer-readable media 1206, and one or more I/O interfaces 1208 that are communicatively coupled, one to another. Although not shown, the computing device 1202 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1204 is representative of functionality to perform one or more operations using hardware. Accordingly, the processing system 1204 is illustrated as including hardware elements 1210 that may be configured as processors, functional blocks, and so forth. This may include implementation in hardware as an application specific integrated circuit or other logic device formed using one or more semiconductors. The hardware elements 1210 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable storage media 1206 is illustrated as including memory/storage 1212. The memory/storage 1212 represents memory/storage capacity associated with one or more computer-readable media. The memory/storage 1212 may include volatile media (such as random-access memory (RAM)) and/or nonvolatile media (such as read only memory (ROM), Flash memory, optical disks, magnetic disks, and so forth). The memory/storage 1212 may include fixed media (e.g., RAM, ROM, a fixed hard drive, and so on) as well as removable media (e.g., flash memory, a removable hard drive, an optical disc, and so forth). The computer-readable media 1206 may be configured in a variety of other ways as further described below.

Input/output interface(s) 1208 are representative of functionality to allow a user to enter commands and information to computing device 1202, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1202 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hard-wired device that performs operations based on hard-wired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 1202. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1202, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1210 and computer-readable media 1206 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1210. The computing device 1202 may be configured to implement particular instructions and/or functions corresponding to the software and/or hardware modules. Accordingly, implementation of a module that is executable by the computing device 1202 as software may be achieved at least partially in hardware, e.g., through use of computer-readable storage media and/or hardware elements 1210 of the processing system 1204. The instructions and/or functions may be executable/operable by one or more articles of manufacture (for example, one or more computing devices 1202 and/or processing systems 1204) to implement techniques, modules, and examples described herein.

The techniques described herein may be supported by various configurations of the computing device 1202 and are not limited to the specific examples of the techniques described herein. This functionality may also be implemented all or in part through use of a distributed system, such as over a “cloud” 1214 via a platform 1216 as described below.

The cloud 1214 includes and/or is representative of the platform 1216 for resources 1218, which are depicted including the sequencing data processor 108. The platform 1216 abstracts underlying functionality of hardware (e.g., servers) and software resources of the cloud 1214. The resources 1218 may include applications and/or data that can be utilized while computer processing is executed on servers that are remote from the computing device 1202. The resources 1218 can also include services provided over the Internet and/or through a subscriber network, such as a cellular or Wi-Fi network.

The platform 1216 may abstract resources and functions to connect the computing device 1202 with other computing devices. The platform 1216 may also serve to abstract scaling of resources to provide a corresponding level of scale to encountered demand for the resources 1218 that are implemented via the platform 1216. Accordingly, in an interconnected device embodiment, implementation of functionality described herein may be distributed throughout the system 1200. For example, the functionality may be implemented in part on the computing device 1202 as well as via the platform 1216 that abstracts the functionality of the cloud 1214.

### CONCLUSION

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. A system comprising:
a sequencing error correction module implemented in a non-transitory computer-readable storage medium and configured to:
train a machine learning model using aligned sequencing reads generated during a nucleic acid sequencing event, the aligned sequencing reads aligned to a reference sequence;
output, by the trained machine learning model, predicted reference sequences for the aligned sequencing reads that have a position of mismatch with the reference sequence; and
selectively correct the aligned sequencing reads according to whether the machine learning model predicts that the aligned sequencing reads are variants or sequencing errors based on the predicted reference sequences relative to the reference sequence.

2. The system of claim 1, wherein, to selectively correct the aligned sequencing reads based on the predicted reference sequence relative to the reference sequence, the sequencing error correction module is further configured to:
update the position of mismatch in a first subset of the aligned sequencing reads in response to the predicted reference sequences matching the reference sequence at the position of mismatch with at least a threshold confidence; and
maintain the position of mismatch in a second subset of the aligned sequencing reads in response to the predicted reference sequences not matching the reference sequence at the position of mismatch with at least the threshold confidence.

3. The system of claim 1, wherein, to train the machine learning model using the aligned sequencing reads generated during the nucleic acid sequencing event, the sequencing error correction module is configured to:
generate training data from the aligned sequencing reads, the training data comprising sequencing reads as model inputs and corresponding reference sequence segments as ground truth outputs;
generate reference sequence predictions from the model inputs using the machine learning model; and
adjust model parameters of the machine learning model based on a loss between the reference sequence predictions and the ground truth outputs.

4. The system of claim 1, wherein the aligned sequencing reads used to train the machine learning model comprise a subset of less than 0.1% of the aligned sequencing reads generated during the nucleic acid sequencing event.

5. The system of claim 1, wherein the machine learning model comprises a neural network.

6. The system of claim 5, wherein the neural network is a recurrent neural network.

7. The system of claim 1, wherein the position of mismatch comprises a substitution, an insertion, or a deletion.

8. The system of claim 1, wherein the aligned sequencing reads comprise long reads, and wherein the sequencing error correction module is further configured to:
subdivide the long reads into smaller read windows of 3000 bases or fewer.

9. The system of claim 1, further comprising:
a variant calling module configured to:
output a variant call based on one or more differences between the selectively corrected aligned sequencing reads and the reference sequence.

10. A method comprising:
receiving a sequencing alignment for a nucleic acid sequencing event, the sequencing alignment comprising a plurality of sequencing reads aligned to a reference sequence;
training a machine learning model to predict a reference sequence segment for respective sequencing reads using, as training data, a subset of the plurality of sequencing reads and the reference sequence; and
generating an error-corrected sequencing alignment based on the reference sequence and outputs of the trained machine learning model.

11. The method of claim 10, wherein training the machine learning model to predict the reference sequence segment for the respective sequencing reads using, as the training data, the subset of the plurality of sequencing reads and the reference sequence comprises:
inputting the subset of the plurality of sequencing reads into the machine learning model;
receiving, as an output of the machine learning model, predicted reference sequence segments for the subset;
comparing the predicted reference sequence segments to corresponding segments of the reference sequence using a loss function; and
adjusting model parameters of the machine learning model in a direction that reduces a loss calculated by the loss function.

12. The method of claim 10, wherein generating the error-corrected sequencing alignment based on the reference sequence and the outputs of the trained machine learning model comprises:
identifying sequencing reads of the plurality of sequencing reads having a mismatched base with the reference sequence;
inputting the identified sequencing reads into the machine learning model;
receiving, as an output of the machine learning model, the predicted reference sequence segment for respective input sequencing reads; and
selectively correcting the mismatched base with the reference sequence based on the predicted reference sequence segment relative to the reference sequence.

13. The method of claim 12, wherein selectively correcting the mismatched base with the reference sequence based on the predicted reference sequence segment relative to the reference sequence comprises:
updating the mismatched base to be the reference sequence in response to the predicted reference sequence segment matching the reference sequence at a position of the mismatched base with at least a threshold probability; or
maintaining the mismatched base in response to the predicted reference sequence segment not matching the reference sequence at the position of the mismatched base with at least the threshold probability.

14. The method of claim 10, wherein the training data exclude sequencing reads aligned to genomic locations having single nucleotide polymorphism sites with a relative population-level frequency above a threshold percentage.

15. The method of claim 10, wherein the machine learning model is trained to predict reference sequence segments that match a corresponding sequence segment of the reference sequence for sequencing reads that have a recurrent sequencing error, the recurrent sequencing error causing a sequence of a respective sequencing read to mismatch with the corresponding sequence segment of the reference sequence.

16. The method of claim 15, wherein the recurrent sequencing error occurs at a same sequence position across multiple sequencing reads of the plurality of sequencing reads.

17. A method comprising:
receiving nucleic acid sequencing data generated for a biological sample, the nucleic acid sequencing data comprising a plurality of sequencing reads;
aligning the plurality of sequencing reads with a reference sequence; and
generating an error-corrected sequence alignment based on nucleotide predictions output by a machine learning model, the machine learning model trained using a subset of the plurality of sequencing reads as model inputs and corresponding reference sequence segments of the reference sequence as ground truth outputs.

18. The method of claim 17, wherein generating the error-corrected sequence alignment based on the nucleotide predictions output by the machine learning model comprises:
identifying a sequencing read of the plurality of sequencing reads having a position of mismatch with the reference sequence;
predicting, via the machine learning model, a reference sequence segment for the sequencing read, the predicted reference sequence segment comprising a sequence of nucleotide predictions; and
comparing a nucleotide prediction of the predicted reference sequence segment to an actual nucleotide of the reference sequence at the position of mismatch.

19. The method of claim 18, wherein generating the error-corrected sequence alignment based on the nucleotide predictions output by the machine learning model further comprises:
correcting the sequencing read to include the actual nucleotide of the reference sequence at the position of mismatch in response to the nucleotide prediction matching the actual nucleotide; or
maintaining a sequenced nucleotide of the sequencing read at the position of mismatch in response to the nucleotide prediction not matching the actual nucleotide.

20. The method of claim 17, wherein generating the error-corrected sequence alignment is further based on probability scores output by the machine learning model, and wherein the machine learning model is further trained using covariates corresponding to the subset of the plurality of sequencing reads as additional model inputs.
